(19) United States
(12) Patent Application Publication
(10) Pub. No.: US 2026/0207321 A1
(43) Pub. Date: Jul. 23, 2026

(54) MEDICAL IMPLANT DELIVERY DEVICES

(71) Applicants: Smith & Nephew, Inc., Memphis, TN (US); Smith & Nephew Asia Pacific Pte. Limited, Singapore (SG)

(72) Inventors: Timothy J. Young, Upton, MA (US); Rick Fu, Randolph, MA (US)

(73) Assignees: Smith & Nephew, Inc., Memphis, TN (US); Smith & Nephew Asia Pacific Pte. Limited, Singapore (SG)

(21) Appl. No.: 19/453,476

(22) Filed: Jan. 20, 2026

Related U.S. Application Data
(60) Provisional application No. 63/747,669, filed on Jan. 21, 2025.

(51) Int. Cl. A61F 2/08; A61F 2/00

(52) U.S. Cl. CPC A61F 2/0805; A61F 2002/0072

(57) ABSTRACT

A medical device delivers an implant by securing, folding, and releasing it through coordinated component movements. The device includes a first component with opposing jaws or a slot for releasably securing the implant in a flat configuration. A second component couples to the first component and moves between positions: a first position maintaining the flat configuration, a second position where it contacts and folds the implant into a folded configuration, and an optional third position allowing the implant to return to flat. The second component both pivots and translates relative to the first component. Movement control utilizes pins with discorectangle profiles extending from the first component that engage compound apertures in the second component, where each aperture has a round portion permitting rotation and a rectilinear portion preventing rotation. Detents and recesses facilitate smooth transitions between portions.

### CROSS REFERENCE TO RELATED APPLICATIONS

This claims the benefit of and priority to US Provisional Patent Application Serial No. 63/747,669, filed on January 21, 2025, the disclosure of which is incorporated herein by reference.

### TECHNICAL FIELD

The present disclosure pertains generally, but not by way of limitation, to orthopedic implants and methods of treatment. More particularly, the present disclosure relates to a tendon repair implant, such as one that is engineered for arthroscopic placement over or in the area of a full or partial thickness tear of the supraspinatus tendon of the shoulder

### BACKGROUND

With its complexity, range of motion and extensive use, a common soft tissue injury is damage to the rotator cuff or rotator cuff tendons. Damage to the rotator cuff is a potentially serious medical condition that may occur during hyperextension, from an acute traumatic tear or from overuse of the joint. Adequate procedures do not exist for repairing a partial thickness tear of less than 50% in the supraspinatus tendon. Current procedures attempt to alleviate impingement or make room for movement of the tendon to prevent further damage and relieve discomfort but do not repair or strengthen the tendon. Use of the still damaged tendon can lead to further damage or injury. There is an ongoing need to deliver and adequately position medical implants during an arthroscopic procedure in order to treat injuries to the rotator cuff, rotator cuff tendons, or other soft tissue or tendon injuries throughout a body.

### BRIEF SUMMARY

This disclosure provides design, material, manufacturing method, and use alternatives for implants used for soft tissue repairs such as but not limited to rotator cuff repairs. An example may be found in a medical device that is adapted to deliver an implant. The medical device includes a first component that is configured to releasably secure the implant, the implant initially in a flat configuration. The medical device includes a second component that is coupled to the first component and that is movable between a first position in which the implant remains in the flat configuration and a second position in which the second component contacts and folds the implant into a folded configuration. A locking mechanism is configured to releasably hold the second component in the second position.

Alternatively or additionally, the second component may be adapted to pivot relative to the first component.

Alternatively or additionally, the second component may be adapted to translate relative to the first component.

Alternatively or additionally, a distal region of the first component may include a first jaw and an opposing second jaw that are configured to secure the implant between the first jaw and the second jaw.

Alternatively or additionally, the first component may include a pin having a discorectangle profile and the second component may include a compound aperture including a round portion that is adapted to allow the pin to rotate therein and a connected rectilinear portion that is adapted to allow the pin to fit therein without rotating.

Alternatively or additionally, the second component may include one or more detents near a junction between the round portion and the connected rectilinear portion.

Alternatively or additionally, the second component may include one or more recesses near the one or more detents that allow the one or more detents to move as the second component is translated and the pin moves between the round portion and the connected rectilinear portion.

Alternatively or additionally, the lock mechanism may include an interaction between the pin and the compound aperture.

Alternatively or additionally, a distal region of the first component may include a slot that is adapted to releasably secure the implant.

Alternatively or additionally, the second component may include an elongate slot and the first component comprises a pin engaged in the elongate slot.

Alternatively or additionally, the second component may be adapted to be slid proximally relative to the first component in order to allow the implant to regain the flat configuration.

Alternatively or additionally, the locking mechanism may include a removable clip that snaps onto the first component and the second component in order to hold the first component relative to the second component.

Another example may be found in a medical device that is adapted to deliver an implant. The medical device includes a first component and a pair of jaws that extend from the first component. The pair of jaws are adapted to releasably secure the implant therein. One or more pins each having a discorectangle profile extend radially outwardly from the first component. The medical device includes a second component that is adapted to pivot and translate relative to the first component. One or more compound apertures are formed in the second component, each of the one or more compound apertures including a round portion adapted to allow a corresponding pin to rotate therein and a connected rectilinear portion adapted to allow the corresponding pin to fit therein without rotating. The second component is free to pivot relative to the first component when each of the pins extend within the round portion of the corresponding compound apertures and the second component is constrained from pivoting relative to the first component when each of the pins extend within the connected rectilinear portion of the corresponding compound apertures.

Alternatively or additionally, the implant may initially be in a flat configuration.

Alternatively or additionally, rotating the second component towards the first component may cause the second component to contact and fold the implant into a folded configuration.

Alternatively or additionally, the second component may form a snap fit within the first component when the second component is pivoted into contact with the implant.

Alternatively or additionally, the first component may include first and second jaws that cooperate to releasably secure the implant.

Another example may be found in a medical device that is adapted to deliver an implant. The medical device includes a first component that is adapted to releasably secure the implant, the implant initially in a flat configuration, and a second component that is coupled to the first component. The second component is movable between a first position in which the implant remains in the flat configuration, a second position in which the second component contacts and folds the implant into a folded configuration, and a third position in which the second component has been translated proximally, thereby allowing the implant to reattain the flat configuration.

Alternatively or additionally, the second component may pivot relative to the first component when moving from the first position to the second position.

Alternatively or additionally, the second component may translate relative to the first component when moving from the second position to the third position.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates a cross-section of an anterior view of a shoulder of a patient;

FIG. 2 shows an example implant;

FIG. 3 illustrates an example implant attached to an example delivery device, with a second component shown in a first position relative to a first component;

FIG. 3A is an enlarged view of the first component of FIG. 3;

FIG. 3B is an enlarged view of the second component of FIG. 3;

FIG. 4 illustrates the portion of the example implant attached to the example delivery device of FIG. 3, with a second component shown in a second position relative to a first component;

FIG. 5 illustrates the portion of the example implant attached to the example delivery device of FIG. 3, with a second component shown locked in the second position relative to a first component;

FIG. 6 illustrates a portion of the example delivery device of FIGS. 3 through 5, showing a snap fit locking mechanism between the first component and the second component;

FIG. 7 illustrates an example implant attached to an example delivery device, with a second component shown in a first position relative to a first component;

FIG. 8 illustrates the example implant attached to the example delivery device of FIG. 7, with a second component shown in a second position relative to a first component;

FIG. 9 illustrates a portion of the example implant attached to the example delivery device of FIG. 7; and

FIG. 10 illustrates the example implant attached to the example delivery device of FIG. 7, with the second component shown in a third position.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

With its complexity, range of motion and extensive use, a common soft tissue injury is damage to the rotator cuff or rotator cuff tendons. Damage to the rotator cuff is a potentially serious medical condition that may occur during hyperextension, from an acute traumatic tear or from overuse of the joint. Current repair procedures may attempt to alleviate impingement or make room for movement of the tendon to prevent further damage and relieve discomfort but do not repair or strengthen the tendon. An accepted treatment for rotator cuff tears may include reattaching the torn tendon to the humeral head using sutures. Additionally, in treating rotator cuff tears, an accepted practice may also include the placement of a scaffold over the repaired tendon to mechanically reinforce the repaired tendon. Therefore, there is an ongoing need to deliver and adequately position medical implants during an arthroscopic procedure in order to treat injuries to the rotator cuff, rotator cuff tendons, or other soft tissue or tendon injuries throughout a body.

In some instances, a medical device is adapted to deliver an implant. The medical device includes a first component that is configured to releasably secure the implant, the implant initially in a flat configuration. The medical device includes a second component that is coupled to the first component and that is movable between a first position in which the implant remains in the flat configuration and a second position in which the second component contacts and folds the implant into a folded configuration. A locking mechanism is configured to releasably hold the second component in the second position.

In some cases, the second component may be adapted to pivot relative to the first component. In some cases, the second component may be adapted to translate relative to the first component. In some cases, a distal region of the first component may include a first jaw and an opposing second jaw that are configured to secure the implant between the first jaw and the second jaw.

In some cases, the first component may include a pin having a discorectangle profile and the second component may include a compound aperture including a round portion that is adapted to allow the pin to rotate therein and a connected rectilinear portion that is adapted to allow the pin to fit therein without rotating. In some cases, the second component may include one or more detents near a junction between the round portion and the connected rectilinear portion. In some cases, the second component may include one or more recesses near the one or more detents that allow the one or more detents to move as the second component is translated and the pin moves between the round portion and the connected rectilinear portion. In some cases, the lock mechanism may include an interaction between the pin and the compound aperture.

In some cases, a distal region of the first component may include a slot that is adapted to releasably secure the implant. In some cases, the second component may include an elongate slot and the first component comprises a pin engaged in the elongate slot. In some cases, the second component may be adapted to be slid proximally relative to the first component in order to allow the implant to regain the flat configuration. In some cases, the locking mechanism may include a removable clip that snaps onto the first component and the second component in order to hold the first component relative to the second component.

In some instances, a medical device is adapted to deliver an implant. The medical device includes a first component and a pair of jaws that extend from the first component. The pair of jaws are adapted to releasably secure the implant therein. One or more pins each having a discorectangle profile extend radially outwardly from the first component. The medical device includes a second component that is adapted to pivot and translate relative to the first component. One or more compound apertures are formed in the second component, each of the one or more compound apertures including a round portion adapted to allow a corresponding pin to rotate therein and a connected rectilinear portion adapted to allow the corresponding pin to fit therein without rotating. The second component is free to pivot relative to the first component when each of the pins extend within the round portion of the corresponding compound apertures and the second component is constrained from pivoting relative to the first component when each of the pins extend within the connected rectilinear portion of the corresponding compound apertures.

In some cases, the implant may initially be in a flat configuration. In some cases, rotating the second component towards the first component may cause the second component to contact and fold the implant into a folded configuration. In some cases, the second component may form a snap fit within the first component when the second component is pivoted into contact with the implant. In some cases, the first component may include first and second jaws that cooperate to releasably secure the implant.

In some instances, a medical device is adapted to deliver an implant. The medical device includes a first component that is adapted to releasably secure the implant, the implant initially in a flat configuration, and a second component that is coupled to the first component. The second component is movable between a first position in which the implant remains in the flat configuration, a second position in which the second component contacts and folds the implant into a folded configuration, and a third position in which the second component has been translated proximally, thereby allowing the implant to reattain the flat configuration.

In some cases, the second component may pivot relative to the first component when moving from the first position to the second position. In some cases, the second component may translate relative to the first component when moving from the second position to the third position.

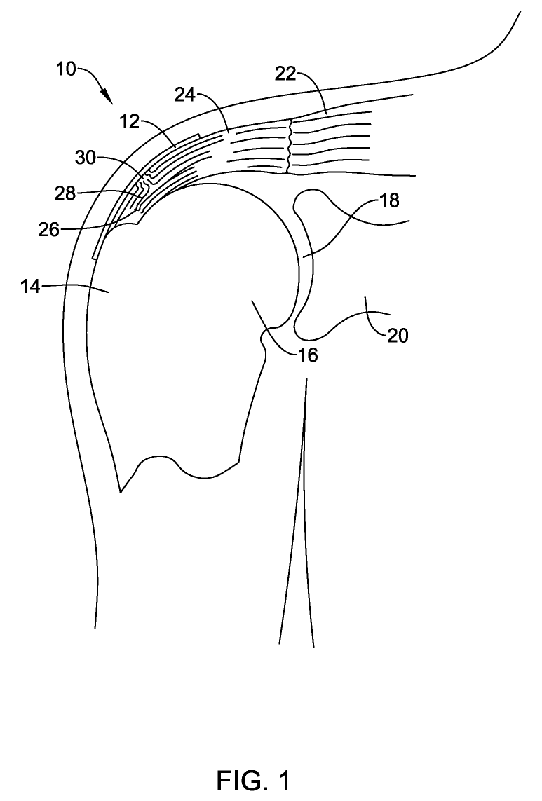

FIG. 1 shows a cross-sectional view of a shoulder 10 including an example bio-compatible implant 12. The shoulder 10 further shows a head 14 of a humerus 16 mating with a glenoid fossa 18 of scapula 20. The glenoid fossa 18 comprises a shallow depression in scapula 20. A supraspinatus tendon 22 is also shown. These muscles (along with others) control the movement of the humerus 16 relative to the scapula 20. A distal tendon 24 of the supraspinatus tendon 22 meets the humerus 16 at an insertion point 26.

In FIG. 1, the distal tendon 24 includes a damaged portion 28 located near the insertion point 26. The damaged portion 28 includes a tear 30 extending within the distal tendon 24. In some cases, the tear 30 may be referred to as a partial thickness tear. In some cases, the tear 30 may be a full thickness tear. In some cases, the tear 30 may be a weakened tendon without a tear. The depicted tear 30 is on the bursal side of the tendon, however, the tear may also be on the opposite or articular side of the distal tendon 24 and/or may include internal tears to the distal tendon 24 not visible on either surface.

FIG. 1 further illustrates that the bio-compatible implant 12 has been placed over the partial thickness tear 30. In this example, the bio-compatible implant 12 is placed on the bursal side of the tendon regardless of whether the tear is on the bursal side, articular side or within the tendon. Further, the bio-compatible implant 12 may overlay multiple tears.

Although one possible treatment site is described in the figures as being located in a shoulder joint, the bio-compatible implant 12 may be used at a variety of different treatment sites, such as the hip, knee, ankle, et. Furthermore, the bio-compatible implant 12 may be used for any of a variety of soft tissue repairs, such as but not limited to the Gluteus Medius, which is a large fan-shaped muscle located in the posterior hip, the Hip Capsule, which is also in the hip. The bio- compatible implant 12 may be used in treating soft tissue injuries in the knee, such as but not limited to ligaments such as the ACL (anterior cruciate ligament), MCL (medial collateral ligament) and the PCL (posterior cruciate ligament) and tendons such as the hamstring tendons, the quadriceps tendon and the patellar tendon. The bio-compatible implant 12 may be used in treating soft tissue injuries in the Achilles tendon. The bio-compatible implant 12 may be used in treating soft tissue injuries for any areas of the body that will accommodate the bio-compatible implant 12.

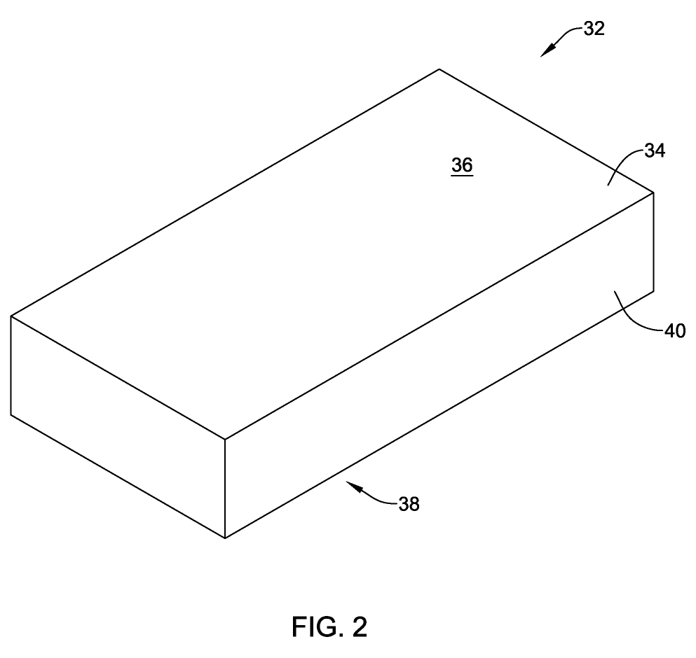

FIG. 2 is a schematic diagram of the example bio-compatible implant 12. The bio-compatible implant 12 may be used for any of a variety of soft tissue repairs, such as but not limited to the Gluteus Medius, which is a large fan-shaped muscle located in the posterior hip, the Hip Capsule, which is also in the hip. The bio-compatible implant 12 may be used in treating soft tissue injuries in the knee, such as but not limited to ligaments such as the ACL (anterior cruciate ligament), MCL (medial collateral ligament) and the PCL (posterior cruciate ligament) and tendons such as the hamstring tendons, the quadriceps tendon and the patellar tendon. The bio-compatible implant 12 may be used in treating soft tissue injuries in the Achilles tendon. The bio-compatible implant 12 may be used in treating soft tissue injuries for any areas of the body that will accommodate the bio-compatible implant 12.

For purposes of illustration, the bio-compatible implant 12 will be described herein with respect to rotator cuff repairs. The bio-compatible implant 12 may be considered as including a scaffold 34. In some instances, the scaffold 34 may define a first primary surface 36 and a second primary surface 38, and a periphery 40 that extends around between the first primary surface 36 and the second primary surface 38.

While drawn as rectilinear, it will be appreciated that this is merely illustrative, as the bio-compatible implant 12 may take any desired shape to best fit at a desired treatment site such as but not limited to a rotator cuff repair. In some cases, the bio-compatible implant 12 may be largely rectilinear, with rounded over corners, for example. In some cases, the bio-compatible implant 12 may be at least partially ovoid. The first primary surface 36 and the second primary surface 38 may be planar or curved. In some cases, the bio-compatible implant 12 may curve around a treatment site. These are just examples.

In some cases, the scaffold 34 may be a collagen scaffold, although other materials are contemplated. In some cases, the scaffold 34 is formed of collagen that has been dehydrated. The scaffold 34 may be formed as a fibrous collection of collagen fibers that define a porous scaffold with a large number of void spaces.

The collagen used to form the scaffold 34 may come from any of a variety of different sources. Collagen is a main structural protein in the extracellular matrix found in various connective tissues and thus can be obtained from various animals. For example, the collagen used to form the scaffold 34 may be bovine-based, i.e., from cows. In some cases, the collagen used to form the scaffold 34 may come from bovine tendon material. As an illustrative but non-limiting example, bovine Achilles tendon is digested down to highly purified collagen, and is reconstituted into a sheet by spinning the fibers around a mandrel. The scaffold 34 may be formed of collagen that has been obtained in other methods as well.

While the scaffold 34 may be formed having a variety of different porosity levels (defined as relative amount of void space to solid material), in some cases the scaffold 34 may have a porosity of at least 50 percent or more, at least 60 percent or more, at least 70 percent or more, or at least 80 percent or more. In some cases, the scaffold 34 may have a porosity of 60 percent to 90 percent, 70 percent to 90 percent, 80 percent to 90 percent, or 85 percent to 90 percent.

Another way to define the scaffold 34 is in terms of average pore size. Pore size refers to a diameter of voids, or empty spaces, formed within the scaffold 34. Average pore size, accordingly, refers to an average diameter of these voids. In some cases, the scaffold 34 may have an average pore size of 20 microns or greater, or 30 microns or greater, or 40 microns or greater, or 50 microns or greater, or 60 microns or greater, or 70 microns or greater, or 80 microns or greater, or 90 microns or greater. In some cases, the scaffold 34 may have an average pore size that is in a range of 100 microns to 500 microns, 100 microns to 400 microns, 100 microns to300microns,100 microns to 150 microns, or 200 microns to 400 microns, for example.

While not expressly shown in FIG. 2, the bio-compatible implant 12 may include a strengthening or reinforcing element that is adapted to provide strength to the soft tissue repair until tissue ingrowth into the bio-compatible implant 12 has occurred. It will be appreciated that the ultimate goal of the bio-compatible implant 12 is to facilitate tissue growth that provides long term strength and functionality to the soft tissue repair. The strengthening or reinforcing element, which may be biosorbable, is merely intended to provide additional strength to the bio-compatible implant 12 immediately after implantation of the bio-compatible implant 12. As the soft-tissue repair gains strength as a result of tissue ingrowth, the strengthening or reinforcing element becomes less important and in some cases may dissolve away.

The bio-compatible implant 12 may be dimensioned in accordance with its desired use. For example, if the bio-compatible implant 12 is intended for use in a rotator cuff repair, the bio-compatible implant 12 may have an overall length of 20 to 45 millimeters (mm) and an overall width of 20 to 40 mm. If the bio-compatible implant 12 is intended for use in an Achilles tendon repair, the bio-compatible implant 12 may have an overall length that is greater than 25 to 45 mm and an overall width that is less than 20 to 30 mm. In some cases, the bio-compatible implant 12 may have an overall thickness that is 1 to 4 mm. In some cases, the bio-compatible implant 12 may have an overall thickness that is about 2 mm. In defining these dimensions, it will be appreciated that the collagen scaffold 34 may not have a uniform thickness throughout the collagen scaffold 34, and the perimeter 40 may not be as evenly defined as shown in the rectilinear representation of FIG. 2. These dimensions may be considered as being averages, for example.

In some instances, delivery of a bio-compatible implant 12 (e.g., a sheet-like implant) to a target site of a patient may require a physician to create an incision in the patient sufficient to access the target implant site. After creating this “access site,” the physician may insert an implant delivery system through the access site and position the distal end of the implant delivery system adjacent the target implant site. The physician may then manipulate the implant delivery system to deploy an implant out of a delivery sheath adjacent the target implant site.

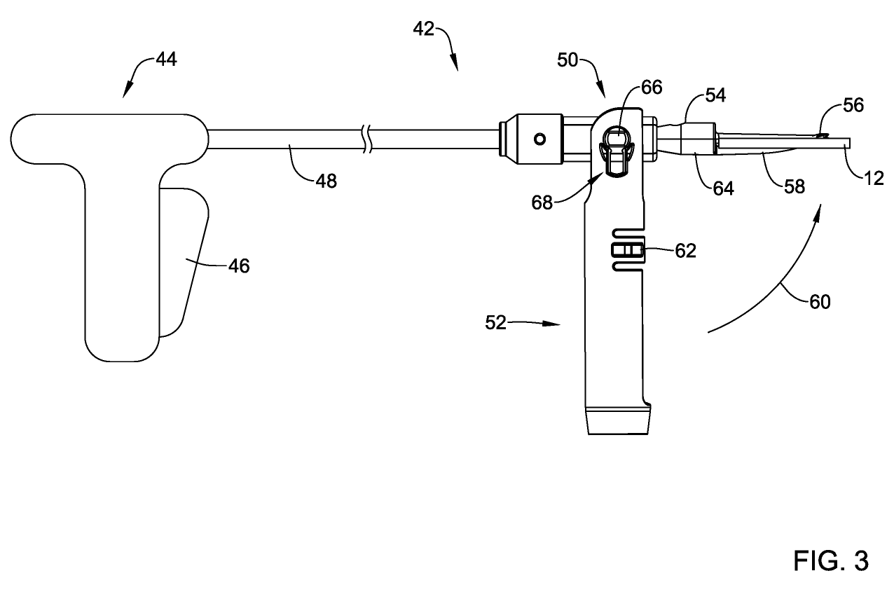

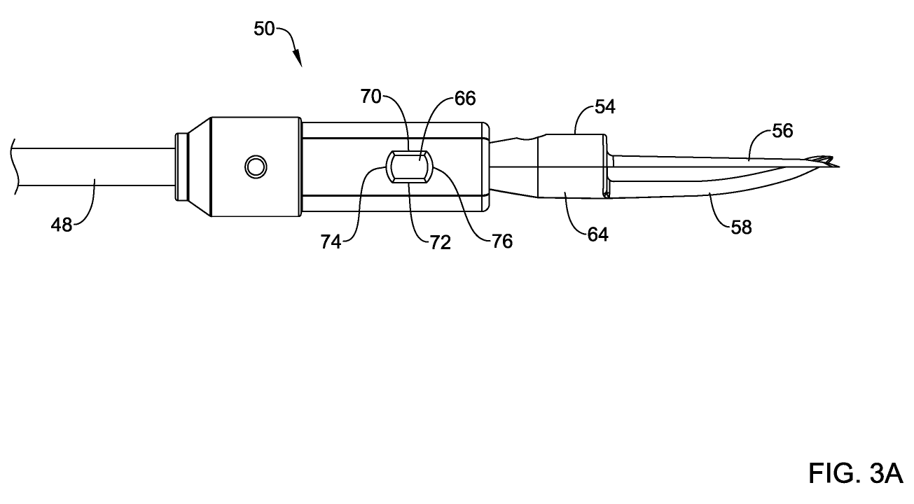

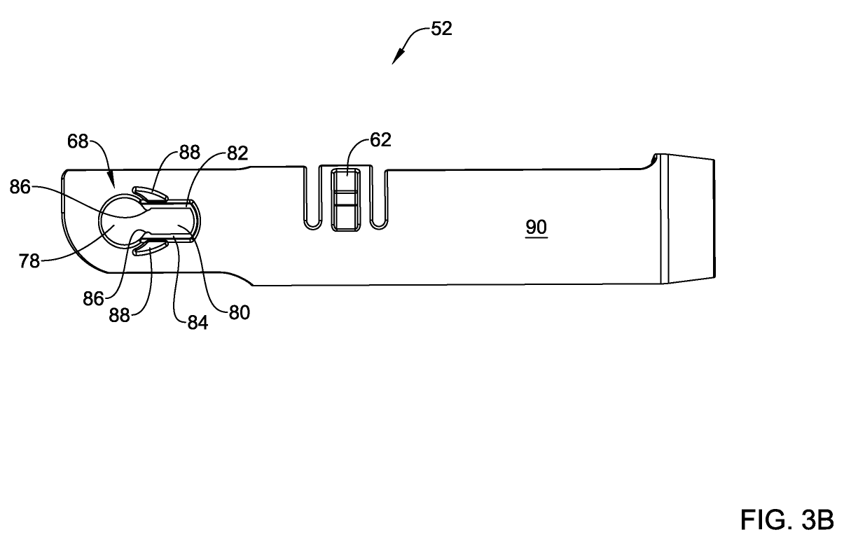

For example, FIG. 3 provides a side view of an example implant delivery system 42 usable for delivering the bio-compatible implant 12. The delivery system 42 includes a handle 44 and a trigger 46 that may be used to actuate the delivery system 42 by squeezing the trigger 46 relative to the handle 44. An elongate shaft 48 extends from the handle 44. In some cases, the delivery system 42 includes a first component 50 and a second component 52. In some cases, the first component 50 may include a body 54. A first jaw 56 and a second jaw 58 may each extend from the body 54, and may be used to releasably secure the bio-compatible implant 12 relative to the body 54. As shown, the bio-compatible implant 12 is initially in a flat configuration. In some cases, the first and second jaws 56 and 58 may include a nitinol component. In some cases, the nitinol component may allow the first and second jaws 56 and 58 to be used to help press the bio-compatible implant 12 flat once deployed.

In some cases, the second component 52 may be movable or pivotable relative to the first component 50. In some cases, as will be shown, the second component 52 may also be translatable relative to the first component 50. In some cases, the second component 52 may be adapted to pivot relative to the first component 50 in a direction indicated by an arrow 60. In some cases, the second component 52 may include a latch mechanism 62 that is adapted to form a snap-fit with a corresponding latch portion 64 of the first component 50. In some cases, the first component 50 may include one or more pins 66 and the second component 52 may include one or more compound apertures 68. The one or more pins 66 may engage with the one or more corresponding compound apertures 68. As an example, the first component 50 may include a pair of pins 66 that are circumferentially 180 degrees apart and the second component 52 may include a pair of compound apertures 68 that are circumferentially 180 degrees apart.

FIG. 3A is an enlarged view of the first component 50 and FIG. 3B is an enlarged view of the second component 52, providing additional details regarding the one or more pins 66 and the one or more compound apertures 68. FIG. 3A shows that each of the one or more pins 66 (only one is visible in this view) may have a discorectangle profile in which each pin 66 has a first straight side 70, a second straight side 72 that is opposite the first straight side 70, a first curved end 74, and a second curved end 76. In some cases, the first curved end 74 forms a semi-circle, i.e., with 180 degrees of curvature. In some cases, the first curved end 74 does not satisfy the requirements of a semi-circle, but forms part of a circle. As an example, the first curved end 74 may include at least 90 degrees of curvature (a quarter of a full circle). In some cases, the second curved end 76 forms a semi-circle, i.e., with 180 degrees of curvature. In some cases, the second curved end 76 does not satisfy the requirements of a semi-circle, but forms part of a circle. As an example, the second curved end 76 may include at least 90 degrees of curvature (a quarter of a full circle).

As seen in FIG. 3B, each of the compound apertures 68 (one is visible in this view) includes a round portion 78 that is adapted to allow a pin 66 to rotate within the round portion 78 as well as a rectilinear portion 80 that allows a pin 66 to translate but does not allow a pin 66 to rotate within the rectilinear portion 80. Thus, the second component 52 is able to rotate or pivot relative to the first component 50 when each pin 66 is disposed within the round portion 78 of the corresponding compound aperture 68. The rectilinear portion 80 includes a first straight edge 82 and an opposing second straight edge 84. The rectilinear portion 80 includes a curved edge 86 that is adapted to match a curvature of the second curved end 76 (of the pin 66). In some cases, an interaction between each of the one or more pins 66 and the corresponding compound apertures 68 provide a locking mechanism that allows the second component 52 to be selectively locked in place relative to the first component 50.

In some cases, the second component 52 includes one or more detents 86 that engage the pins 66. In some cases, the second component 52 may be moved proximally relative to the first component 50, urging each pin 66 into the rectilinear portion 80 of the compound aperture 68. In some cases, one or more recesses 88 are formed within the second component 52 near to where the detents 86 are formed. In some cases, this allows the detents 86 to flex or temporarily move out of the way, allowing each pin 66 to move into the rectilinear portion 80 of each compound aperture 68. In some cases, the second component 52 includes a body portion 90 that accommodates the bio-compatible implant 12 therein.

As noted, the bio-compatible implant 12 is in a flat configuration as shown in FIG. 3. In some cases, the bio-compatible implant 12 may be pre-loaded. In some cases, the bio-compatible implant 12 may be loaded by the user at the time of surgery. Once the bio-compatible implant 12 is loaded, rotating or pivoting the second component 52 up towards the first component 50, in the direction indicated by the arrow 60 in FIG. 3, causes the body portion 90 of the second component 52 to contact either edge of the bio-compatible implant 12, thereby causing the bio-compatible implant 12 to fold into a folded configuration in which the bio-compatible implant 12 has a U-shape (if viewed from an end of the bio-compatible implant 12). In some cases, the bio- compatible implant 12 may be hydrated before folding the bio-compatible implant 12 into a folded configuration having a U-shape. In some cases, the bio-compatible implant 12, still within the first and second jaws 56 and 58 of the first component 50, may be submerged in a saline bath in order to hydrate the bio-compatible implant 12 and make the bio-compatible implant 12 more compliant.

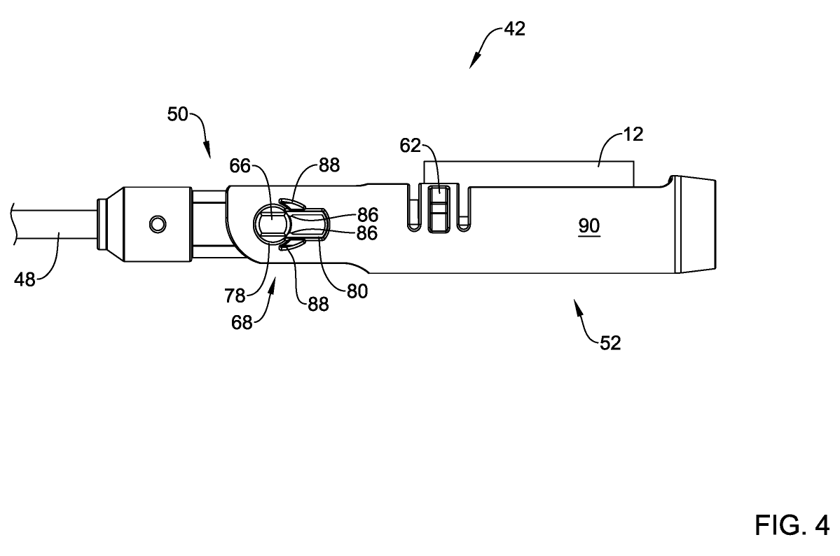

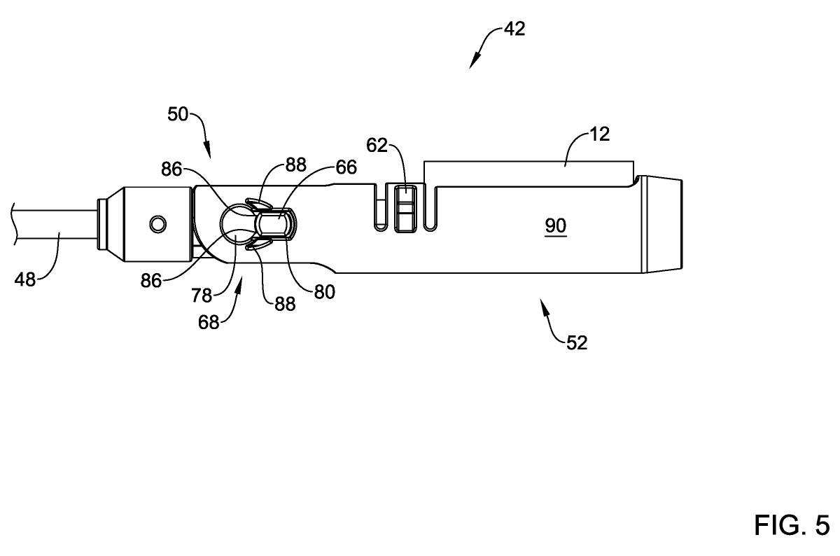

FIG. 4 shows the second component 52 pivoted up into a second position in which the second component 52 may be considered as being coaxial with the first component 50. In FIG. 4, each of the pins 66 (one is visible) remains disposed within the round portion 78 of each of the compound apertures 68. In FIG. 5, each of the pins 66 are now disposed within the rectilinear portion 80 of each of the compound apertures 68. In this position, the second component 52 is locked relative to the first component 50 and cannot easily or accidently be moved out of this arrangement. In some cases, the first component 50 and the second component 52 may together function as a cannula or sheath for insertion of the bio-compatible implant 12.

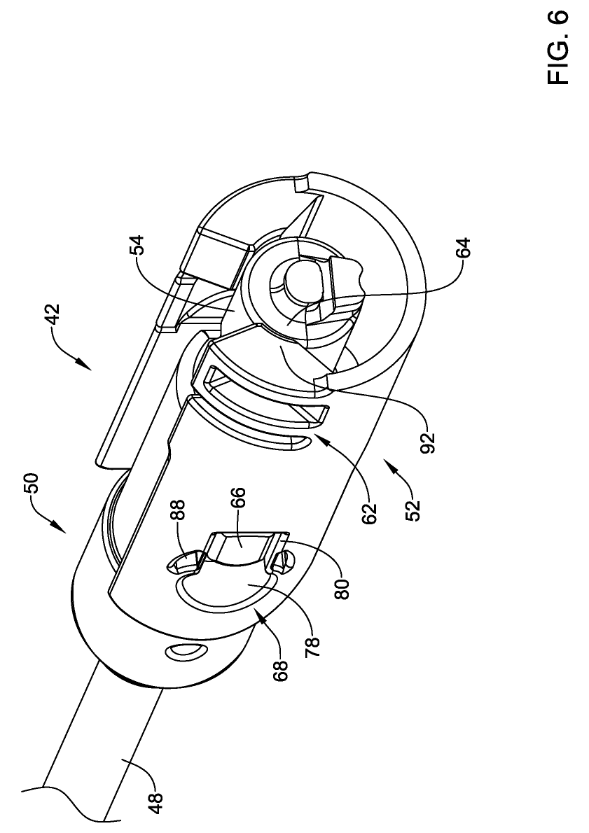

FIG. 6 is a cutaway view of the delivery device 42, with the body portion 90 of the second component 52 removed. The latch mechanism 62 includes a region 92 that engages with the latch portion 64 of the body 54. The latch mechanism 62 provides a secondary locking mechanism that helps to hold the second component 52 in position relative to the first component 50 regardless of whether each of the pins 66 are in the round portion 78 or the rectilinear portion 80 of the compound apertures 68. In some cases, the latch mechanism 62 and the latch portion 64 of the body 54 help to prevent rotational unfolding of the first component 50 relative to the second component 52 during introduction of the delivery device 42 into a joint space. In some cases, the first component 50 may have a tendency to rotate away from the second component 52, absent a locking mechanism, as a result of axial forces applied to the delivery device 42. In some cases, a funnel may alternatively be used to assemble the bio-compatible implant 12 into an open cannula for delivery.

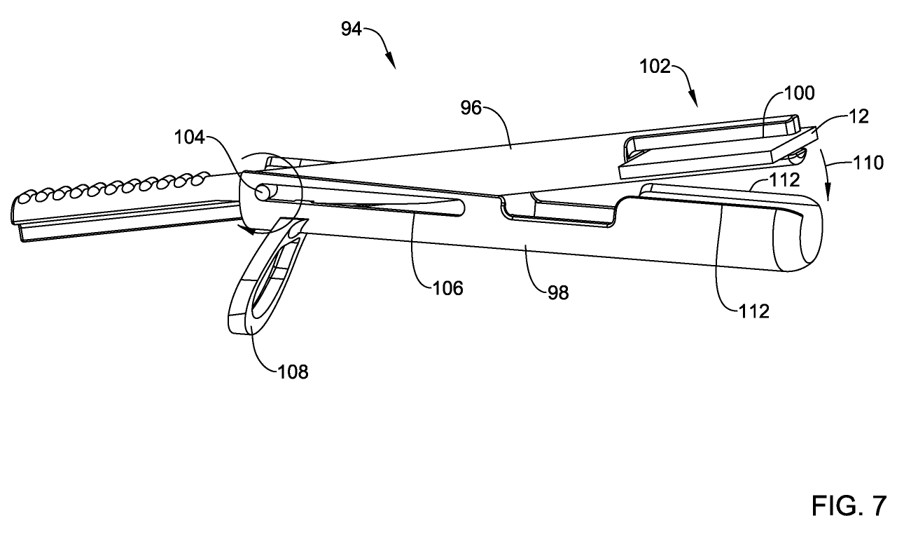

FIG. 7 provides a perspective view of an example delivery device 94 usable for delivering the bio-compatible implant 12. The delivery device 94 includes a first component 96 and a second component 98. The first component 96 includes a slot 100 that is formed within a distal region 102 of the first component 96. In some cases, the slot 100 is adapted to releasably secure the bio-compatible implant 12 in a flat configuration. The first component 96 includes one or more pins 104 (one pin 104 is visible in this view). The second component 98 includes one or more elongate slots 106 (one elongate slot 106) is visible in this view. In some cases, the first component 96 may include a pair of pins 104 that are circumferentially 180 degrees apart and the second component 98 may include a pair of elongate slots 106 that are circumferentially 180 degrees apart. In some cases, the delivery device 94 may include a locking mechanism 108 that may be used to hold the first component 96 and the second component 98 together. In some cases, the first component 96 may be held in position relative to the second component 98 by holding the delivery device 94 in a user’s hand. In some cases, rotational unfolding of the first component 96 relative to the second component 98 is less of an issue, since the rotational point is more proximal in the delivery device 94 (relative to the rotational point in the delivery device 42).

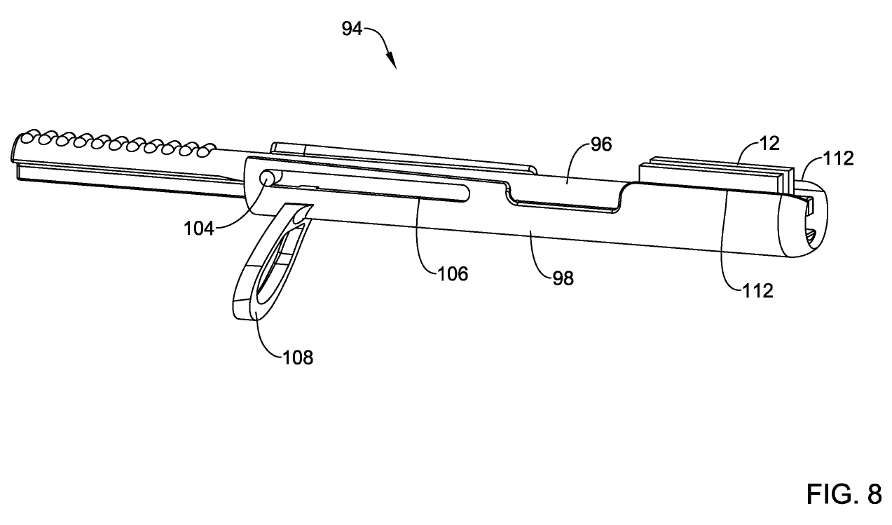

In some cases, the bio-compatible implant 12 may be hydrated before folding the bio-compatible implant 12 into a folded configuration having a U-shape. In some cases, the bio-compatible implant 12, still within the slot 100 of the first component 96, may be submerged in a saline bath in order to hydrate the bio-compatible implant 12 and make the bio-compatible implant 12 more compliant. As seen in FIG. 7, the bio-compatible implant 12 beings in a flat configuration. Urging the first component 96 in a direction indicated by an arrow 110 causes the bio-compatible implant 12 to contact side walls 112 of the second component 98, thereby causing the bio-compatible implant 12 to fold into a folded configuration in which the bio-compatible implant 12 has a U-shape. This can be seen in FIG. 8.

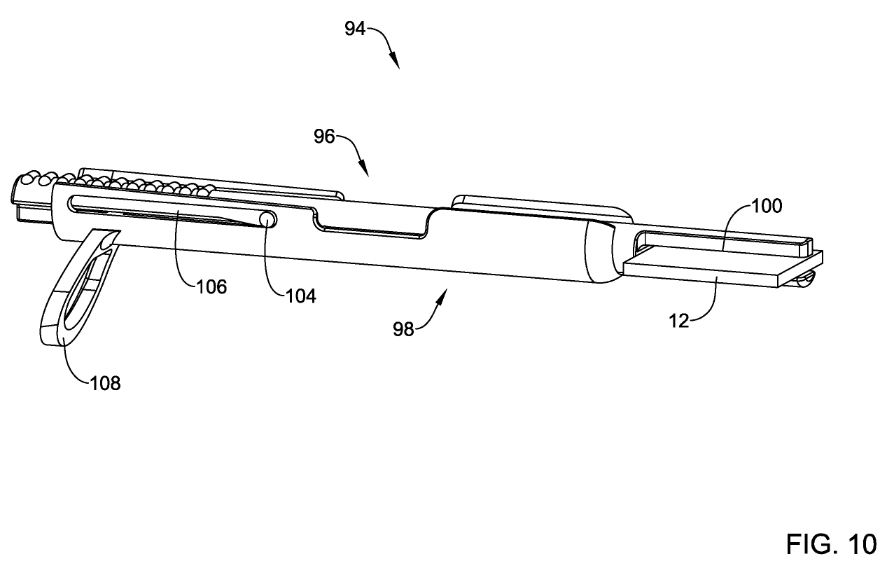

FIG. 7 may be considered as showing the second component 98 in a first position in which the bio-compatible implant 12 remains flat. FIG. 8 may be considered as showing the second component 98 in a second position in which the bio-compatible implant 12 has been folded by virtue of contact with the second component 98. FIG. 10 may be considered as showing the second component 98 in a third position in which the first component 96 has moved distally relative to the second component 98, thereby allowing the bio-compatible implant 12 to regain its flat configuration for implantation. In comparing FIG. 8 and FIG. 10, it can be seen that in FIG. 10, the pin 104 has translated within the elongate slot 106. In some cases, the second component 98 may pivot relative to the first component 96 when moving from the first position to the second position. In some cases, the second component 98 translates relative to the first component 96 when moving from the second position to the third position.

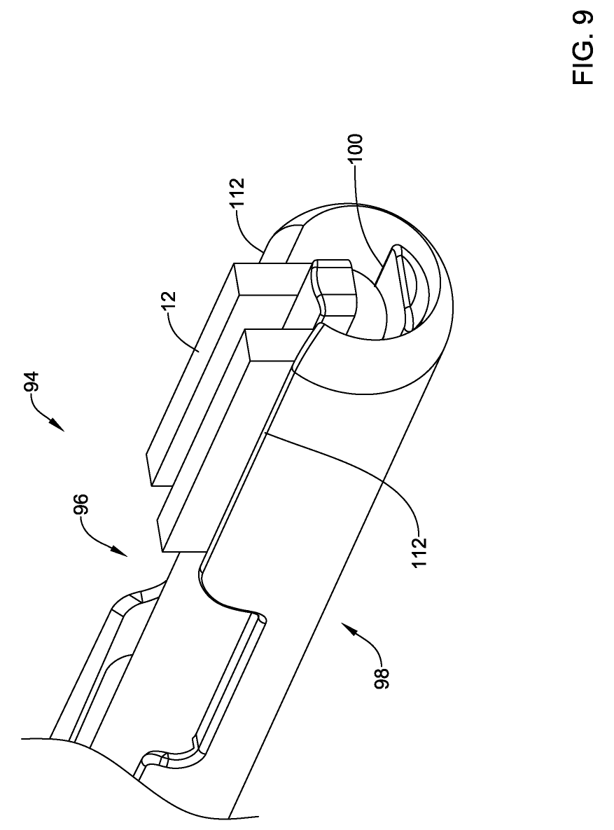

FIG. 9 is a perspective view of a portion of the delivery device 94, showing how the bio-compatible implant 12 remains within the slot 100 of the first component 96 and how the bio-compatible implant 12 forms a U-shape folded configuration as a result of the side walls 112 contacting the bio-compatible implant 12 when the first component 96 is moved towards the second component 98 in a direction indicated by the arrow 110. In some cases, the side walls 112 may be dimensioned to be longer than the bio-compatible implant 12 in order to limit the bio-compatible implant 12 accidently snagging on the delivery device 94. In some cases, the side walls 112 have some flexibility and are able to flex outwardly in order to allow the first component 96 to fit within the second component 98. In some cases, either the bio-compatible implant 12 or the side walls 112 may be dimensioned such that the bio-compatible implant 12 is at least largely covered by the side walls 112. In some cases, if part of the bio-compatible implant 12 sticks out above the side walls 112, that part of the bio-compatible implant 12 may conform to the skin incision and move out of the way during introduction into a joint space.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure’s scope is, of course, defined in the language in which the appended claims are expressed.

What is claimed is:

1. A medical device adapted to deliver an implant, the medical device comprising:
a first component configured to releasably secure the implant, the implant initially in a flat configuration;
a second component that is coupled to the first component, the second component movable between a first position in which the implant remains in the flat configuration and a second position in which the second component contacts and folds the implant into a folded configuration; and
a locking mechanism configured to releasably hold the second component in the second position.

2. The medical device of claim 1, wherein the second component is adapted to pivot relative to the first component.

3. The medical device of claim 1, wherein the second component is adapted to translate relative to the first component.

4. The medical device of claim 1, wherein a distal region of the first component includes a first jaw and an opposing second jaw that are adapted to secure the implant between the first jaw and the second jaw.

5. The medical device of claim 1, wherein the first component comprises a pin having a discorectangle profile and the second component comprises a compound aperture including a round portion adapted to allow the pin to rotate therein and a connected rectilinear portion adapted to allow the pin to fit therein without rotating.

6. The medical device of claim 1, wherein the second component comprises one or more detents near a junction between the round portion and the connected rectilinear portion.

7. The medical device of claim 6, wherein the second component comprises one or more recesses near the one or more detents that allow the one or more detents to move as the second component is translated and the pin moves between the round portion and the connected rectilinear portion.

8. The medical device of claim 5, wherein the lock mechanism comprises an interaction between the pin and the compound aperture.

9. The medical device of claim 1, wherein a distal region of the first component comprises a slot that is adapted to releasably secure the implant.

10. The medical device of claim 1, wherein the second component comprises an elongate slot and the first component comprises a pin engaged in the elongate slot.

11. The medical device of claim 1, wherein the second component is configured to be slid proximally relative to the first component in order to allow the implant to regain the flat configuration.

12. The medical device of claim 1, wherein the locking mechanism comprises a removable clip that snaps onto the first component and the second component in order to hold the first component relative to the second component.

13. A medical device adapted to deliver an implant, the medical device comprising:
a first component;
a pair of jaws extending from the first component, the pair of jaws configured to releasably secure the implant therein;
one or more pins extending radially outwardly from the first component, each of the one or more pins having a discorectangle profile;
a second component that is adapted to pivot and translate relative to the first component;
one or more compound apertures formed in the second component, each of the one or more compound apertures including a round portion adapted to allow a corresponding pin to rotate therein and a connected rectilinear portion adapted to allow the corresponding pin to fit therein without rotating;
wherein the second component is free to pivot relative to the first component when each of the pins extend within the round portion of the corresponding compound apertures and the second component is constrained from pivoting relative to the first component when each of the pins extend within the connected rectilinear portion of the corresponding compound apertures.

14. The medical device of claim 13, wherein the scaffold is initially in a flat configuration.

15. The medical device of claim 14, wherein rotating the second component towards the first component causes the second component to contact and fold the scaffold into a folded configuration.

16. The medical device of claim 15, wherein the second component forms a snap fit within the first component when the second component is pivoted into contact with the scaffold.

17. The medical device of claim 13, wherein the first component comprises first and second jaws that cooperate to releasably secure the implant.

18. A medical device configured to deliver an implant, the medical device comprising:
a first component adapted to releasably secure the implant, the implant initially in a flat configuration;
a second component that is coupled to the first component, the second component movable between:
a first position in which the implant remains in the flat configuration;
a second position in which the second component contacts and folds the implant into a folded configuration; and
a third position in which the second component has been translated proximally, thereby allowing the scaffold to reattain the flat configuration.

19. The medical device of claim 18, wherein the second component pivots relative to the first component when moving from the first position to the second position.

20. The medical device of claim 18, wherein the second component translates relative to the first component when moving from the second position to the third position.
